(19) United States
(12) Patent Application Publication
(10) Pub. No.: US 2003/0087265 A1
(43) Pub. Date: May 8, 2003

(54) Specific microarrays for breast cancer screening

(76) Inventors: Edward Sauter, Columbia, MO (US); Garrett Dubois, Trappe, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 10/151,847

(22) Filed: May 20, 2002

Related U.S. Application Data
(63) Continuation-in-part of application No. 09/765,791, filed on Jan. 19, 2001.
(60) Provisional application No. 60/177,273, filed on Jan. 21, 2000.
(60) Provisional application No. 60/292,149, filed on May 18, 2001.
(60) Provisional application No. 60/340,320, filed on Dec. 14, 2001.
(60) Provisional application No. 60/340,153, filed on Dec. 14, 2001.

(51) Int. Cl. C12Q 1/68; C12M 1/34

(52) U.S. Cl. 435/6; 435/287.2

(57) ABSTRACT

Microarrays specific for breast cancer are provided. Also provided are methods for detecting breast cancer in patients or screening therapeutics for the treatment or prevention of breast cancer by analyzing expression levels of specific genes in BECs or quantifying specific protein levels in breast ductal fluid.

### CONTINUING APPLICATION DATA

This is a Continuation in Part of application Ser. No. 09/765,791, filed Jan. 19, 2001, which claims priority to a Provisional Application No. 60/177,273, filed Jan. 21, 2000. This application also claims priority under 35 U.S.C. §119 based upon U.S. Provisional Patent Application No. 60/292,149 filed May 18, 2001, U.S. Provisional Patent Application No. 60/340,320 filed Dec. 14, 2001, and U.S. Provisional Patent Application No. 60/340,153 filed Dec. 14, 2001.

### FIELD OF THE INVENTION

The present invention generally relates to the fields of molecular biology and oncology; to microarrays specific for detecting biomarkers associated with breast cancer; to methods of detecting breast cancer by analyzing biomarkers present in breast ductal fluid samples; and to methods of determining efficacy of therapeutics in treating or preventing breast cancer by analyzing biomarkers present in breast ductal fluid samples.

### BACKGROUND OF THE INVENTION

Breast cancer is the most common noncutaneous cancer among women in the United States. Over forty-one thousand women in the U.S. die yearly from the disease. The only well-established procedures to screen subjects for breast cancer are physical examination and mammography. Unfortunately, physical examination does not identify a significant number of early breast cancers, and mammograms miss 10-40% of early breast cancers. (Giuliano, A. E., The Breast, In: Current Surgical Diagnosis & Treatment, 293-316, 1994). Moreover, in the recently operated breast, mammography and breast examination are generally of little help in predicting residual disease.

A number of pathogenic factors in the diagnostic biopsy have been associated with residual disease/local failure in the breast, including positive margins, gross multicentricity, extensive intraductal carcinoma, an age under 35-40, and invasive lobular carcinoma. (Lagios, M. D., Semin Surg Onc, 8: 122-28, 1992; Harris, J. R. et al, Cancer of the Breast, In: Cancer: Principles and Practice of Oncology 4^{th} ed., 1264-1332, 1993). Despite the currently available markers, approximately half of the women who undergo mastectomy for presumed residual disease will not have disease when the breast is investigated microscopically.

Although the early detection of breast cancer will lead to a higher cure rate, the ideal form of treatment is prevention. The prevention of breast cancer is hindered by the difficulty in identifying an effective agent. Effective agents are difficult to identify in part because of the long period required for breast cancer to develop and, consequently, the requirement for lengthy clinical trials to test the efficacy of the agent, if the end point is the prevention of cancer. One way to shorten the time required to find an effective agent is the identification of intermediate biomarkers, which are biological alterations in cells or tissues that occur between the time of initiation and tumor invasion. An agent that partially or completely reverses the intermediate biomarker back to a normal phenotype may be interrupting carcinogenesis. Evaluating the effect of the agent requires the analysis of tissue, cells, or non-cellular fluid.

Abbreviations

“BEC” means “breast epithelial cell.”

“BP3-FR” means “fragmented insulin-like growth factor binding protein-3.”

“CART” means “classification and regression trees.”

“DCIS” means “ductal carcinoma in situ.”

“IC” means “invasive carcinoma.”

“IGF-1” means “insulin-like growth factor-1.”

“IGFBP-3” means “IGF binding protein-3.”

“IMAC” means “immobilized metal affinity capture.”

“LCM” means “laser capture microdissection.”

“LCR” means “ligase chain reaction.”

“NAF” means “nipple aspirate fluid.”

“PCR” means “polymerase chain reaction.”

“PSA” means “prostate-specific antigen.”

“SELDI” means “surface-enhance laser desorption/ionization.”

“SELEX” means “Systematic Evolution of Ligands by EXponential enrichment”.

“TOF” means “time-of-flight.”

Definitions

The term “adsorb” refers to the detectable binding between an absorbent and an analyte either before or after washing with an eluant (selectivity threshold modifier).

The term “adsorbent” refers to any material capable of adsorbing an analyte. The term “adsorbent” is used herein to refer both to a single material (“monoplex adsorbent,” e.g., a compound or functional group) to which the analyte is exposed and to a plurality of different materials (“multiplex adsorbent”) to which a sample is exposed. The adsorbent materials in a multiplex adsorbent are referred to as “adsorbent species.” For example, an addressable location on a substrate can comprise a multiplex adsorbent characterized by many different adsorbent species (e.g., anion exchange materials, metal chelators, or antibodies), having different binding characteristics.

The term “antibody” refers to a polypeptide ligand substantially encoded by an immunoglobulin gene or immunoglobulin genes, or fragments thereof, which specifically binds and recognizes an epitope (e.g., an antigen).

The recognized immunoglobulin genes include the kappa and lambda light chain constant region genes; the α, γ, δ, ε and μ heavy chain constant region genes; and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, and humanized antibodies. “Fc” portion of an antibody refers to that portion of an immunoglobulin heavy chain that comprises one or more heavy chain constant region domains, CH₁, CH₂, and CH₃, but does not include the heavy chain variable region.

The term “apoptosis related genes” as used herein includes, but is not limited to, bcl-2, bax, TRPM-2, TIPM-1, TIPM-2, ICE genes, c-myc, p53, K-ras, COX-1, COX-2, MDM-2, p21, p27, p16/INK4, PCNA, H-ras, MMP-1, ERK-1, ERK-2, and NFk-B.

The term “aptamers” as used herein refer to non-naturally occurring nucleic acid ligand having specific binding affinity for a target molecule. The target molecule is a three dimensional chemical structure other than a polynucleotide that binds to the nucleic acid through a mechanism which predominantly depends on Watson/Crick base pairing or triple helix binding. The aptamer, or nucleic acid ligand, is not a nucleic acid having a known physiological function of being bound by the target molecule; and the target molecule is a protein, such as, nucleic acid polymerase, bacteriophage coat protein, serine protease, mammalian receptor, mammalian hormone, mammalian growth factor, ribosomal protein, and viral rev protein. Molecules of any size can serve as targets. U.S. Pat. Nos. 5,270,163 and 5,475,096 describes the Systematic Evolution of Ligands by EXponential enrichment (SELEX) method for identifying aptamers that specifically bind to target molecules.

The terms “deoxyribonucleic acid” and “DNA” as used herein refer to a polymer composed of deoxyribonucleotides.

The term “eluant” refers to an agent, typically a solution, that is used to mediate adsorption of a protein to an adsorbent. Eluants also are referred to as “selectivity threshold modifiers.”

The term “elution conditions” refers to the elution characteristics to which a target protein is exposed.

The terms “genes associated with breast cancer” or “breast cancer related genes” as used herein refer to genes, the expression level of which is associated with the pathology of breast cancer. They include, but are not limited to, apoptosis related genes, growth factor system genes, and other key genes of interest.

The term “insulin-like growth factor/human kallikrein system genes” as used herein includes, but is not limited to, IGF-1, IGFBP-1, -2, -2, -4, -5, -6, -7, -8; IGFR-1, -2; IGF II;, PSA; hK2; hK6; and hK10.

The terms “nucleic acid” or “nucleic acid molecule” refer to a deoxyribonucleotide or ribonucleotide polymer in either single- or double-stranded form, and unless otherwise limited, would encompass known analogs of natural nucleotides that can function in a similar manner as naturally occurring nucleotides.

The term “oligonucleotide” as used herein denotes single-stranded nucleotide multimers of from 2 to about 500 nucleotides in length.

The term “oligopeptide” as used herein refers to peptides with fewer than about 10 to 20 residues, i.e. amino acid monomeric units.

The term “other key genes of interest” as used herein includes, but is not limited to, estradiol; estrone; 17α-OH progesterone; MMP2; MMP9; erbB-1; HER-2/neu; hTERT; erbB-2; VEGF; PGE₂; hst-1; KDR; int-2; cyclin D1, 2, 3; CEA; H-ras; K-ras; and mammoglobin.

The term “peptide” as used herein refers to any compound produced by amide formation between a carboxyl group of one amino acid and an amino group of another amino acid.

The term “plurality” means at least two.

The term “polynucleotide” as used herein refers to single- or double-stranded polymers composed of nucleotide monomers of generally greater than 100 nucleotides in length.

The term “polypeptide” as used herein refers to peptides with more than 10 to 20 residues.

The term “protein” as used herein refers to polypeptides of specific sequence of more than about 50 residues.

The terms “proteins associated with breast cancer” or “breast cancer related proteins” as used herein refer to proteins, the quantity of which present in breast ductal fluid is associated with the pathology of breast cancer. They include, but are not limited to, erbB-1; HER-2/neu; hTERT; erbB-2; hst-1; KDR; int-2; cyclin D1, 2, 3; CEA; H-ras; K-ras; mammoglobin; PSA; IGFBP-1, -2, -4, -5, -6; hK2; IGFBP-3; and p53.

The terms “ribonucleic acid” and “RNA” as used herein refer to a polymer composed of ribonucleotides.

The term “selectivity conditions” refers to the selectivity characteristics to which a target protein is exposed.

### BRIEF DESCRIPTION OF THE FIGURES

FIG. 1. Diagram depicting a microarray.

FIG. 2. Photomicrographs illustrating that the technique of LCM is able to selectively (outlined area) collect BECs without disrupting surrounding foam cells.

FIG. 3. 5-20 kDa SELDI-TOF profiles of 8 NAF specimens from subjects with breast cancer (top 4 profiles) and from subjects without breast cancer (bottom 4 profiles).

FIG. 4. NAF box plots in cancer patients (cancer) and in subjects free of cancer (non-cancer) overall and by menopausal status: (A) PSA in log scale (for each data point 0.5 has been added to avoid log 0); (B) IGFBP-3. The median (middle line), 25 and 75th percentiles (lower and upper boundaries of the box, respectively), and lowest and highest data within 1.5 times the 25-75th percentiles (lower and upper hatch lines) are illustrated. Points more extreme are shown by individual plot symbols. IGFBP-3=340.7 and 205.3 are out of bounds and not plotted.

FIG. 5. Serum box plots in cancer patients (cancer) and in subjects free of cancer (non-cancer) overall and by menopausal status: (A) PSA in log scale (for each data point 0.5 has been added to avoid log 0); (B) BP3-FR. The median (middle line), 25 and 75th percentiles (lower and upper boundaries of the box, respectively), and lowest and highest data within 1.5 times the 25-75th percentiles (lower and upper hatch lines) are illustrated. Points more extreme are shown by individual plot symbols. BP3-FR=6.1 is out of bounds and not plotted.

FIG. 6. NAF plots demonstrating the odds of breast cancer for (A) PSA and (B) IGFBP-3 (reference values are the medians in the non-cancer group).

### DETAILED DESCRIPTION OF THE INVENTION

The breast ducts of adult non-pregnant, non-lactating women secrete small amounts of fluid. (Keynes, G., Br J Surg, 11: 89-121, 1923) This fluid does not escape because the nipple ducts are occluded by smooth muscle contraction, dried secretions, and keratinized epithelium. Breast ductal fluid can contains several types of cells, including exfoliated BECs. Because breast cancer develops from ductal and lobular epithelium, the breast ductal fluid is a potentially useful epidemiologic and clinical research tool. Moreover, a wide array of proteins are secreted into the breast ductal fluid. These secreted proteins are highly concentrated and organ specific. For example, secreted prostate specific antigen (PSA) is present in nipple aspirate fluid (NAF) at levels 100-1000-fold higher than in female serum.

Breast ductal fluid can be obtained through aspiration of the nipple with a modified breast pump (Petrakis N. L. et al, J Natl Cancer Inst, 54: 829-34, 1975). The breast ductal fluid obtained by nipple aspirate is called nipple aspirate fluid (NAF). Breast ductal fluid also can be obtained through fiberoptic endoscopy of the breast (ductoscopy), a minimally invasive diagnostic technology. Ductoscopy provides a more cellular mammary ductal irrigation that is specific to the area of concern and is termed mammary ductal irrigation.

The present invention provides microarrays of binding agents, such as polynucleotides and peptides, and the use thereof, to analyze breast ductal fluid or BECs obtained therefrom, thereby detecting breast cancer and identifying therapeutics for the treatment or prevention of breast cancer. The present invention also provides methods for analyzing protein profiles in breast ductal fluid of subjects with or without breast cancer using surface-enhance laser desorption/ionization time-of-flight (SELDI-TOF) mass spectrometry, in order to identify proteins associated with breast cancer, detecting breast cancer, and screening for therapeutics for the treatment or prevention of breast cancer.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

Genes and Proteins Associated with Breast Cancer

Many disease states are characterized by alterations in the expression levels of various genes through either changes in the copy number of the genetic DNA or changes in the level of transcription (e.g., through control of initiation, provision of RNA precursors, RNA processing, etc.) of particular genes. Therefore, control of the cell cycle and cell development, as well as diseases, is characterized by variations in the transcription levels of particular genes. A number of genes in BECs have been identified as having an important role in breast cancer. Oncogenes or tumor repressor genes for breast tumor phenotyping include those involved in apoptosis, development and differentiation of breast ductules/alveoli, and the early response pathway. By way of example but not limitation, Table 1 provides a list of genes that are associated with breast cancer. Thus, detection of elevated or reduced expression levels of these characteristic oncogenes or tumor repressor genes provides an effective diagnostic of breast cancer.

TABLE 1
Genes Associated with Breast Cancer

| | | | | | |
|---|---|---|---|---|---|
| Apoptosis-Related Genes | | | | | |
| Bcl-2 | TRPM-2 | TIPM-1, -2 | COX-1, -2 | p27 | ERK-1, -2 |
| ICE genes | bax | K-ras | MDM-2 | p16/INK4 | NFk-B |
| c-myc | p53 | H-ras | p21 | PCNA | |
| Growth Factor System Genes | | | | | |

| IGF-1 | | hK2 |
|---|---|---|
| IGFBP -1, -2, -3, -4, -5, -6, -7, -8 | | hK6 |
| IGFR-1, -2 | | hK10 |
| IGF II | | PSA |
| Other Key Genes of Interest | | |
| Estradiol | Estrone | 17α-OH progesterone |
| MMP2 | MMP9 | erbB-1 |
| HER-2/new | hTERT | erbB-2 |
| VEGF | PGE2 | hst-1 |
| KDR | int-2 | cyclin D1, 2, 3 |

Changes at the mRNA level, however, are not necessarily proportional to changes at the protein level because of differences in rates of protein translation and degradation. Furthermore, it is the protein and not the mRNA that provides cellular function, whether it be for communication, metabolism or building cellular architecture. Breast ductal fluid, including NAF and mammary ductal irrigation, contains a wide range of proteins secreted by BECs, including those proteins that are associated with breast cancer. By way of example, but not limitation, Table 2 provides a list of proteins associated with breast cancer that are present in breast ductal fluid. Thus, measuring changes in the levels of these breast cancer related proteins allows one to effectively detect breast cancer in patients and screen for therapeutics for the treatment or prevention of breast cancer.

TABLE 2
Proteins Associated with Breast Cancer

| | | | |
|---|---|---|---|
| PSA | IGFBP-1, -2, -4, -5, -6 | erbB-1 | int-2 |
| hK2 | IGFBP-3 | HER-2/neu | KDR |
| p53 | mammoglobin | hTERT | CEA |
| erbB-2 | cyclin D1, 2, 3 | hst-1 | H-ras |
| K-ras | | | |

Microarrays

The microarrays of the present invention have a plurality of biopolymeric probes stably associated with a surface of a solid support. The biopolymeric probes of the subject microarrays are oligonucleotides, such as deoxyribonucleic acids, ribonucleic acids, and the like, or peptides, such as antibodies (e.g., polyclonal, monoclonal and binding fragments thereof), peptides with high affinity to the targets, as well as analogues and mimetics thereof, ligands, receptors, and the like, or aptamers, such as DNA or RNA aptamers that specifically bind to protein targets. The biopolymeric probes may be obtained from a natural source or synthesized using available technologies.

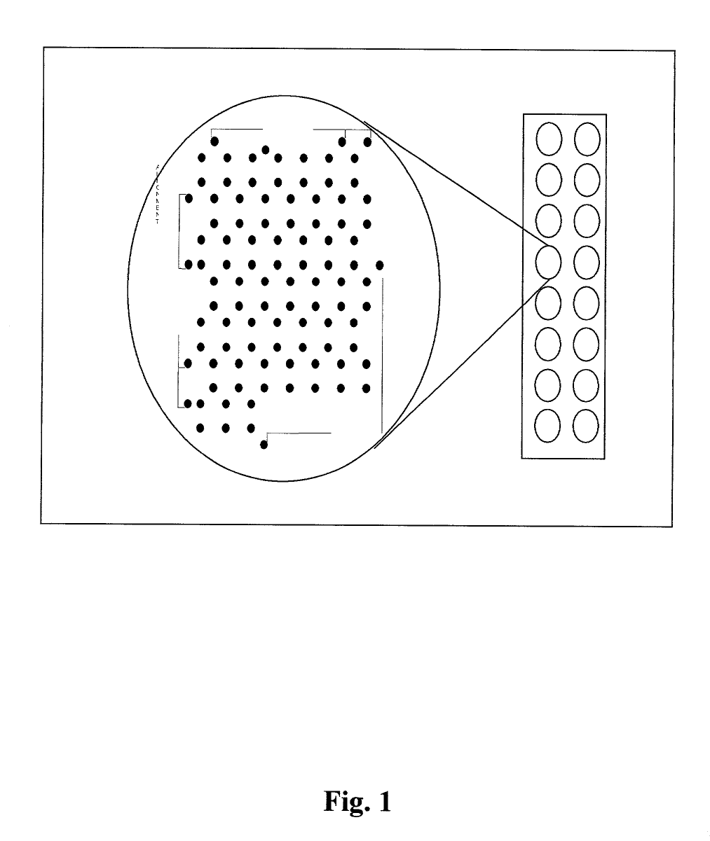

The probe spots on the microarray may be any convenient shape, but will typically be circular, elliptoid, oval, annular, or some other analogously curved shape where the shape may be a result of the particular method employed to produce the microarray. The density of the probe positions, including calibration and control probes, on the surface of the support is selected to provide for adequate resolution of binding events, where the density will generally range from about 8-200 probes/microarray to about 3000 probes/microarray. The probe positions may be arranged in any convenient pattern across or over the surface of the microarray, such as in rows and columns so as to form a grid, in a circular pattern, and the like, where generally the pattern of positions will be present in the form of a grid across the surface of the solid support. In the microarrays of the subject invention, a single pattern of spots may be present on the microarray or the microarray may comprise a plurality of different probe position patterns, each pattern being as defined above. When a plurality of probe position patterns are employed, the patterns may be identical to each other or different. (FIG. 1)

In the subject microarrays, the probes are immobilized on the surface of a solid support. By immobilized it is meant that the probes maintain their position relative to the surface of the solid support under hybridization and washing conditions. As such, the probes can be non-covalently or covalently stably associated with the support surface. Examples of non-covalent association include non-specific adsorption, specific binding through a specific binding pair member covalently attached to the support surface, and entrapment in a matrix material, e.g. hybridization, to occur. Examples of covalent binding include covalent bonds formed between the target and a functional group present on the support surface, e.g. —OH, where the functional group may be naturally occurring or present as a member of an introduced linking group.

The solid substrate of the subject microarrays may be fabricated from a variety of materials. The materials from which the substrate is fabricated ideally should exhibit a low level of non-specific binding of target during hybridization or specific binding events. Specific materials of interest include: glass, plastics, metals, nylon, nitrocellulose, polypropylene, etc. The configuration of the substrate of the subject microarrays may take a variety of configurations depending on the intended use of the microarray. Generally, an overall rectangular configuration is preferred.

The solid substrate of the subject microarrays comprises at least one surface on which a pattern of probe molecules is present, where the surface may be smooth or substantially planar, or have irregularities, such as depressions or elevations. The surface on which the pattern of probes is presented may be modified with one or more different layers of compounds that serve to modulate the properties of the surface in a desirable manner. The microarrays of the subject invention may be incorporated into a structure that provides for ease of analysis, high throughput, or other advantages, such as in a biochip format, a multiwell format, etc.

Biopolymeric Probes

A specific feature of the subject microarrays is that at least one of the biopolymeric probes of the microarray corresponds to a gene or a protein associated with breast cancer. More particularly, in the case when the biopolymeric probes of the microarray are oligonucleotide probes, at least one of the oligonucleotide probes is complementary to the mRNA transcripts of a breast cancer related gene or nucleic acids derived from the mRNA transcripts. In the case when the biopolymeric probes of the microarray are protein-capture agents, such as peptides or aptamers, at least one of the protein-capture agent selectively binds to a breast cancer related protein. Candidate breast cancer related genes and proteins are listed in Table 1 and Table 2.

The subject microarrays typically comprise one or more additional probes that do not correspond to breast cancer related genes or proteins of interest. Other probes that serve as control genes, the function of which is to ensure the quality of the data, also might be present on the substrate surface of the microarray. These control probes may be oligonucleotide probes that are complementary to specific housekeeping genes of interest, such as β-actin and GAPDH, or pecific external, unrelated genes of interest, such as yeast genome, arabidopsis, and human 18s rRNA sequences. These control probes also may be protein-capture agents that specifically bind to proteins such as those found in all cells or physiologic fluids, including, but not limited to, β-actin and IgG.

Array Preparation

Microarrays may be prepared using methods known in the art. The solid substrate or support can be fabricated according to known procedures, where the particular means of fabrication depends on the material from which the support is made. The pattern of probe molecules is then prepared and stably associated with the surface of the support. The probe molecules may be isolated from cells, tissues, or organisms using standard techniques. Such methods typically involve tissue/cell homogenization, nucleic acid/protein extraction, chromatography, centrifugation, affinity binding, etc. The probe molecules may be further treated in order to improve hybridization and detection or enhance association with the surface of the support. Such treatments might include: reverse transcription, nuclease treatment, DNA amplification, etc.

Following stable placement of the pattern of probe molecules on the support surface, the resultant microarray may be used as is or incorporated into a biochip, multiwell or other device for use in a variety of binding applications.

Sample Analysis

Sample analysis using the subject microarrays generally involves the following steps: 1) preparation of the target; 2) contact of the target with the array under conditions sufficient for the target to bind with the corresponding probe; 3) removal of any unbound target; and 4) imaging.

Target preparation depends on the specific nature of the target, e.g., whether the target is nucleic or peptidic. A variety of different protocols may be used to generate labeled biopolymeric targets for detection in the imaging step. Labels may be either directly detectable, such as isotopic and fluorescent moieties incorporated into a moiety of the target, or detectable through combined action, such as labels that provide for a signal only when the target with which they are associated is specifically bound to a probe molecule. In one embodiment, total RNA is isolated and amplified, where necessary, and labeled with radioactive probe.

Following its preparation, the labeled target solution is contacted with the microarray under conditions sufficient for binding between the targets in the sample and the probes on the microarray, where such conditions can be adjusted, as desired, to provide for an optimum level of specificity in view of the particular assay being performed. The method of achieving contact depends on the configuration of the array. Generally, the target sample will be a fluid sample and contact will be achieved by introduction of an appropriate volume of the fluid sample onto the microarray surface, where introduction is via an inlet port, deposition, dipping the microarray into a fluid sample, etc. Contact of the target solution and the microarray must be maintained for a period of time sufficient for binding between the targets and the probes to occur. Such time will vary depending on the nature of the targets and the probes. In one embodiment, the labeled targets are incubated with the microarray so that the target sequences hybridize to complementary polynucleotide probes of the microarray. Incubation conditions can be adjusted so that hybridization occurs with precise complementary matches or with various degrees of less complementarity.

Following binding of probe and target, the nonhybridized labeled targets are removed from the support surface by washing. The resultant hybridization patterns of labeled target on the surface of the microarray may be visualized or detected in a variety of ways, with the particular manner of detection being dependent upon the particular label of the target. Representative detection methods include scintillation counting, fluorescence measurement, calorimetric measurement, radioactive probe quantification, etc. Hybridization patterns can be compared to identify differences between patterns. Where microarrays in which each of the different probes correspond to a known gene, any discrepancies can be related to a differential expression of a particular gene in the sources being compared. In certain embodiments, after removal of the nonhybridized probes, a scanner is used to determine the levels and patterns of signal. The scanned images are examined to determine the degree of complementarity and the relative abundance of each polynucleotide sequence on the microarray.

As such, the subject microarrays can find use in a variety of applications, including profiling differential gene expression in BECs and discovering potential therapeutic and diagnostic drug targets. The findings can be used both to screen for new or recurrent disease or to evaluate response to therapy, as with a chemotherapeutic or chemopreventive agent.

Kits

Also provided for are kits for performing binding assays using the subject microarrays, where kits for carrying out differential gene expression analysis assays are preferred. Such kits according to the subject invention will at least comprise a microarray according to the subject invention, where the microarray may simply comprise a pattern of target molecules on a planar support or be incorporated into a multiwell configuration, biochip configuration, or other configuration. The kits may further comprise one or more additional reagents for use in the assay to be performed with the microarray, where such reagents include: probe generation reagents, reagents used in the binding step, signal producing system members, etc.

Monitoring Gene Expression

The present invention provides oligonucleotide microarrays specific for breast cancer related genes and the use thereof for detecting breast cancer or screening therapeutics for the treatment or prevention of breast cancer.

Probe Composition

One of skill in the art will appreciate that an enormous number of array designs are suitable for the practice of this invention. The oligonucleotide microarray will typically include a number of probes that specifically hybridize to the nucleic acid, the expression of which is related to breast cancer. In addition, in certain embodiments, the array will include one or more control probes.

Test Probes. In one embodiment, the oligonucleotide microarray includes “test probes.” These are oligonucleotides that have sequences complementary to particular subsequences of the genes whose expressions are related to breast cancer. Thus, the test probes are capable of specifically hybridizing to the target nucleic acid they are to detect. In one embodiment, the oligonucleotide microarray of the subject invention will include genes associated with the induction or inhibition of apoptosis: bcl-2, bax, TRPM-2, TIPM-1, TIPM-2, ICE genes, c-myc, p53, K-ras, COX-1, COX-2, MDM-2, p21, p27, p16/INK4, PCNA, H-ras, MMP-1, ERK-1, ERK-2, and NFk-B. In another embodiment, the subject microarray will include genes involved in the insulin-like growth factor system/human kallikrein system: IGF-1; IGFBP-1, -2, -2, -4, -5, -6, -7, -8; IGFR-1, -2; IGF II, PSA; hK2, hK6, and hK10. In yet another embodiment, all of the genes listed in Table 1 are present on the microarray.

Control Probes. In addition to test probes that bind the target nucleic acid(s) of interest, the oligonucleotide microarray can contain a number of control probes, the function of which is to ensure the quality of the data. Control probes that might be present on the substrate surface include internal housekeeping genes, external unrelated genes, and the like. Specific housekeeping genes of interest include: β-actin and GAPDH. Specific external, unrelated genes of interest include: yeast genome, arabidopsis, and human 18s rRNA sequences.

Nucleic Acid Sample

One of skill in the art will appreciate that in order to measure the transcription level (and thereby the expression level) of a gene or genes, it is desirable to provide a nucleic acid sample comprising mRNA transcript(s) of the gene or genes, or nucleic acids derived from the mRNA transcript(s). As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Samples suitable for use in the instant invention include, but are not limited to, mRNA transcripts of the gene or genes, cDNA reverse transcribed from the mRNA, cRNA transcribed from the cDNA, DNA amplified from the genes, RNA transcribed from amplified DNA, and the like.

In a particular embodiment, such a nucleic acid sample is the total mRNA isolated from a population of BECs obtained from breast ductal fluid. The nucleic acid (either genomic DNA or mRNA) may be isolated from the population of BECs according to any of a number of methods well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization with Nucleic Acid Probes, Part I. Theory and Nucleic Acid Preparation, P. Tijssen, ed. Elsevier, N.Y. (1993)).

In one particular embodiment, the total nucleic acid is isolated from a population of BECs using, for example, an acid guanidinium-phenol-chloroform extraction method and polyA⁺ mRNA is isolated by oligo dT column chromatography or by using (dT)ₙ magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

In certain embodiments, the nucleic acid sample is amplified prior to hybridization. One of skill in the art will appreciate that whatever amplification method is used, if a quantitative result is desired, care must be taken to use a method that maintains or controls for the relative frequencies of the amplified nucleic acids.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The oligonucleotide microarray may then include probes specific to the internal standard for quantification of the amplified nucleic acid.

Other suitable amplification methods include, but are not limited to, PCR (Innis, et al., PCR Protocols. A Guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988), and Barringer, et al., Gene, 89: 117 (1990)), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87:1874 (1990)).

In a specific embodiment, the sample mRNA is reverse transcribed with a reverse transcriptase and a primer consisting of oligo dT and a sequence encoding the phage T7 promoter to provide a single-stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro polymerization are well known to those of skill in the art (see, e.g., Sambrook et al., Molecular Cloning: A Laboratory Manual (2nd ed.), Vols. 1-3, Cold Spring Harbor Laboratory, (1989)) and this particular method is described in detail by Van Gelder, et al., Proc. Natl. Acad. Sci. USA, 87: 1663-1667 (1990) who demonstrate that in vitro amplification according to this method preserves the relative frequencies of the various RNA transcripts. Moreover, Eberwine et al. Proc. Natl. Acad. Sci. USA, 89: 3010-3014 provides a protocol that uses two rounds of amplification via in vitro transcription to achieve greater than 10⁶ fold amplification of the original starting material thereby permitting expression monitoring even where biological samples are limited.

It will be appreciated by one of skill in the art that the direct transcription method described above provides an antisense RNA (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

The protocols cited above include methods of generating pools of either sense or antisense nucleic acids. Indeed, one approach can be used to generate either sense or antisense nucleic acids as desired. For example, the cDNA can be directionally cloned into a vector (e.g., Stratagene's pBluscript 11 KS(+) phagemid) such that it is flanked by the T3 and T7 promoters. In vitro transcription with T3 polymerase will produce RNA of one sense (the sense depending on the orientation of the insert), while in vitro transcription with T7 polymerase will produce RNA having the opposite sense. Other suitable cloning systems include phage lamda vectors designed for Cre-IoxP plasmid subcloning (see e.g., Palazzolo et al., Gene, 88: 25-36 (1990)).

### Detecting Breast Cancer and Screening Therapeutics

Monitoring expression levels of a breast cancer related gene(s) in BECs allows one to detect breast cancer in a patient and screen therapeutics for the treatment or prevention of breast cancer. For example, where breast cancer is to be detected in a patient, the expression levels of one or more breast cancer related genes in BECs from the patient is compared to that from a non-cancer subject and any deviations in the expression levels of the breast cancer related gene(s) in the BECs from the patient as compared to that from the non-cancer subject indicates breast cancer.

Similarly, where the efficacy of a therapeutic for treating or preventing breast cancer is to be determined, the therapeutic will be administered to a patient having breast cancer. Nucleic acids from the BECs obtained from the patient's breast ductal fluid are isolated as described above, hybridized to an oligonucleotide microarray containing oligonucleotide probes directed to one or more breast cancer related genes, and the expression levels of the breast cancer related gene(s) are determined. Any changes in the expression levels of the breast cancer related gene(s) compared to that prior to the administration of the therapeutic indicates the efficacy of the therapeutic in treating or preventing breast cancer.

Quantifying Protein Levels

The present invention also provides microarrys specific for detecting and quantifying breast cancer related proteins and the use thereof for detecting breast cancer or screening therapeutics for the treatment or prevention of breast cancer.

Probe Composition

The subject microarray will typically include a number of protein-capture agents that specifically bind to proteins, the quantification of which is associated with breast cancer. In addition, in certain embodiments, the array will include one or more control probes.

Test Probes. In one embodiment, the subject microarray includes “test probes.” These are protein-capture agents that hybridize specifically to the proteins that are related to breast cancer. Such protein-capture agents are antibodies (e.g., polyclonal, monoclonal and binding fragments thereof), peptides with high affinity to the targets, as well as analogues and mimetics thereof, ligands, receptors, aptamers (e.g., DNA or RNA aptamers), and the like. In one embodiment, at least one of the protein-capture agents specifically binds to one of the proteins listed on Table 2.

Control Probes. In addition to test probes that bind the target protein(s) of interest, the subject microarray can contain a number of control probes, the function of which is to ensure the quality of the data. Control probes that might be present on the substrate surface include, but are not limited to, those protein-capture agents that specifically bind to β-actin, IgG, IgM, and albumin.

Protein Sample

Methods of obtaining samples containing proteins from an organism are know to those skilled in the art. In a specific embodiment of the present invention, samples containing proteins are obtained from breast ductal fluid. Breast ductal fluid can be obtained through aspiration of the nipple with a modified breast pump (Petrakis N. L. et al., J Natl Cancer Inst, 54:829-34, 1975). Breast ductal fluid that is obtained in this manner is referred to as NAF. Breast ductal fluid also can be obtained through ductoscopy. The protein samples of the present invention are obtained by ridding of cells from the breast ductal fluid using means that are known to those skilled in the art. The protein sample also may be further purified or labeled prior to analysis.

Detecting Breast Cancer and Screening Therapeutics

Quantifying a breast cancer related protein(s) in breast ductal fluid allows one to detect breast cancer in a patient and screen therapeutics for the treatment or prevention of breast cancer. For example, where breast cancer is to be detected in a patient, the quantity of one or more breast cancer related proteins in breast ductal fluid from the patient is compared to that from a non-cancer subject and any differences in the quantity of the breast cancer related protein(s) in the breast ductal fluid from the patient as compared to that from the non-cancer subject indicates breast cancer.

Similarly, where the effects of a therapeutic for treating or preventing breast cancer is to be determined, the therapeutic will be administered to a patient having breast cancer. Protein samples from the breast ductal fluid of the patient are isolated as described above, hybridized to a microarray containing protein-capture agents directed to one or more breast cancer related proteins and the quantity of the breast cancer related protein(s) are determined. Any changes in quantity of the breast cancer related protein(s) compared to that prior to the administration of the therapeutic indicates the efficacy of the therapeutic in treating or preventing breast cancer.

Surface-Enhanced Laser Desorption/Ionization Time-of-Flight (SELDI-TOF) Mass Spectrometer

Surface-enhanced laser desorption/lonization (SELDI) was invented by Hutchens and Yip (U.S. Pat. No. 5,719,060). When coupled to a time-of-flight mass spectrometer (TOF), the SELDI system provides a means for rapidly detecting and accurately calculating the mass of compounds ranging from small molecules and peptides of less than 1000 Da, up to proteins of 500 kDa or more based on measured time-of-flight. More specifically, a SELDI system comprises a proteinchip array that captures proteins in a sample and a laser desorption/ionization time-of-flight mass spectrometry that analyzes the proteins by their molecular weights.

Proteinchip arrays provide a variety of selectivity conditions that allow one to optimize protein capture and analysis. Each selectivity condition is defined by an adsorbent and an eluant, wherein the adsorbent is positioned on the surface of the solid support of the proteinchip array. Adsorbents that exhibit grossly different binding characteristics typically differ in their bases of attraction or mode of interaction. The basis of attraction is generally a function of chemical or biological molecular recognition. Bases for attraction between an adsorbent and a protein include, for example, (1) a salt-promoted interaction, e.g., hydrophobic interactions, thiophilic interactions, and immobilized dye interactions; (2) hydrogen bonding and/or van der Waals forces interactions and charge transfer interactions, such as in the case of a hydrophilic interactions; (3) electrostatic interactions, such as an ionic charge interaction, particularly positive or negative ionic charge interactions; (4) the ability of the protein to form coordinate covalent bonds (i.e., coordination complex formation) with a metal ion on the adsorbent; (5) enzyme-active site binding; (6) reversible covalent interactions, for example, disulfide exchange interactions; (7) glycoprotein interactions; (8) biospecific interactions; or (9) combinations of two or more of the foregoing modes of interaction. That is, the adsorbent can exhibit two or more bases of attraction, and thus be known as a “mixed functionality” adsorbent. Other bases for attraction between an adsorbent and a protein also can be antibody-antigen, DNA-protein, receptor-ligand, and other molecular interactions.

The eluants, or wash solutions, selectively modify the threshold of absorption between the protein and the adsorbent. The ability of an eluant to desorb and elute a bound protein is a function of its elution characteristics. Different eluants can exhibit grossly different elution characteristics, somewhat different elution characteristics, or subtly different elution characteristics. As in the case of adsorbents, eluants that exhibit grossly different elution characteristics generally differ in their basis of attraction to the protein. Examples of different eluants are: pH-based, ionic strength-based, water structure-based, detergent-based, hydrophobicity-based eluants, or combinations of two or more of the above.

Advantages of SELDI-TOF systems include low limit of detection, as low as 1 fmole of protein; small sample size, 0.5-500 μl; and significant results, accurate molecular weight. Development in SELDI-TOF technology has been the subject of a number of U.S. patents, such as U.S. Pat. Nos. 6,225,047 and 6,294,790. A variety of commercially available SELDI-TOF systems also are being used by those skilled in the art for analyzing protein profiles in certain sample(s), e.g., SELDI-TOF-MS ProteinChip® System from Ciphergen, Fremont, Calif.

The present invention provides methods for identifying proteins associated with breast cancer by analyzing protein profiles in breast ductal fluid using a SELDI-TOF system. The breast ductal fluid may be NAF obtained by nipple aspiration or mammary ductal irrigation obtained by ductoscopy. The breast cancer related proteins in breast ductal fluid are identified by: 1) obtaining a sample from mammary ductal fluid of a patient having breast cancer; 2) providing a proteinchip array having a selectivity condition, wherein the selectivity condition is defined by an adsorbent and an eluant; 3) contacting said sample to said proteinchip array; 4) washing said proteinchip array with said eluant; and 5) detecting and quantifying proteins retained by said proteinchip array by a laser desorption/ionization time-of-flight mass spectrometer, wherein one or more of said proteins that are present differentially on said proteinchip array when compared to that from a non-cancer subject are identified as protein(s) associated with breast cancer.

The present invention also provides methods for detecting breast cancer in a patient by analyzing protein profiles in the patient's breast ductal fluid using SELDI-TOF system. The breast ductal fluid may be NAF obtained by nipple aspiration or mammary ductal irrigation obtained by ductoscopy. Proteins associated with breast cancer are identified by the methods described above. A protein associated with breast cancer may be present or over abundant in a patient having breast cancer as compared to a non-cancer subject, or absent or reduced in a patient having breast cancer as compared to a non-cancer subject. Breast cancer in a patient is detected by: 1) obtaining a sample from mammary ductal fluid of said patient, wherein said sample comprises at least one breast cancer related protein; 2) providing a proteinchip array having a selectivity condition, wherein the selectivity condition is defined by an adsorbent and an eluant, wherein the selectivity condition allows said at least one breast cancer related protein to be retained by said proteinchip array; 3) contacting said sample to said proteinchip array; 4) washing said proteinchip array with said eluant; 5) detecting and quantifying said at least one breast cancer related protein retained by said proteinchip array by a laser desorption/ionization time-of-flight mass spectrometer; and 6) comparing quantities of said at least one breast cancer related protein retained by said proteinchip array to those from a non-cancer patient, wherein any differences in quantities indicate breast cancer in said patient.

Further provided by the present invention are methods for screening therapeutics in treating or preventing breast cancer: 1) administering an appropriate amount of said therapeutic to a patient having breast cancer; 2) obtaining a sample from mammary ductal fluid of said patient, wherein said sample comprises at least one breast cancer related protein; 3) providing a proteinchip array having a selectivity condition, wherein the selectivity condition is defined by an adsorbent and an eluant, wherein the selectivity condition allows said at least one breast cancer related protein be retained by said proteinchip array; 4) contacting said sample to said proteinchip array; 5) washing said proteinchip array with said eluant; 6) detecting and quantifying said at least one breast cancer related protein retained by said proteinchip array by a laser desorption/ionization time-of-flight mass spectrometer; and 7) comparing quantities of said at least one breast cancer related protein retained by said proteinchip array with those measured prior to the administration of said therapeutic, wherein any differences in quantities indicate the efficacy of said therapeutic.

### EXAMPLES

The following examples are offered by way of illustration and not by way of limitation.

Breast Ductal Fluid Specimen Collection

Breast ductal fluid may be obtained through aspiration of the nipple with a modified breast pump or ductoscopy.

NAF Specimen Collection

NAF was obtained using a modified breast pump. The device consists of a 10-ml syringe attached to the end of a no. 4 endotracheal tube over which is placed a respiratory humidification adapter. Subjects were seated in a comfortable position, and the breast nipple was cleansed with alcohol. After the alcohol evaporated, a warm, moist cloth was placed on each breast. After 1-2 minutes, the cloths were removed, the patient compressed her breast with both hands, and the plunger of the syringe was withdrawn to the 7-ml level and held for 15 seconds or until the patient experienced discomfort. Fluid in the form of droplets was collected in capillary tubes, with samples from each breast collected separately. The quantity of fluid varied from 1 to 200 μl.

Occasionally, keratin plugs rather than NAF were obtained after suction was completed. The plugs were removed with an alcohol swab and suctioning repeated. At times, suctioning had to be performed two or three times to remove all of the plugs. To obtain additional fluid, the nipple was gently compressed between two fingers. One or two additional droplets of fluid often appeared.

For subjects with invasive cancer, the mastectomy specimen was aspirated immediately after removal from the chest wall. Aspiration of mastectomy specimens was performed in a fashion similar to aspiration of the intact breast, with the exception that warm cloths were not used on the mastectomy specimens.

Mammary Ductal Irrigation and/or Brushing Specimen Collection

Mammary ductal irrigation involved gently compressing the subject's nipple, which produced dried skin cells or breast fluid. The nipple then was gently dilated with graduated lacrimal duct probes to a diameter of 1.2 mm. The ductoscope was attached to the camera and light cords. The insufflation syringe was attached to the working channel, and a white sponge was used to white balance the screen. The ductoscope was passed into the dilated nipple orifice. Gentle traction was applied to the nipple-areolar complex with one hand while the other hand guided the scope through the lumen. Using air insufflation, the scope was passed through the mammary ducts under direct visualization. If the screen became clouded, saline was injected through the working channel to clear the view. Once the lesion was in view, the saline and discharge was aspirated into a collection device and then rinsed into containers holding 3% polyethylene glycol in denatured alcohol (Shandon Lipshaw, Pittsburgh, Pa.).

A very thin wire probe with spiral grooves at the tip was used to retrieve samples through the scope. A brush also might be used in retrieving the samples. The samples were removed under direct vision. The location of the duct orifice and character of the duct lining was recorded both as the scope was advanced and as it was removed. The depth at which each irrigation and/or brushing sample was collected also was recorded.

Gene Expression Profile Analysis Using cDNA Microarrays

Sample Preparation for mRNA Targets

Laser Capture Microdissection (LCM)

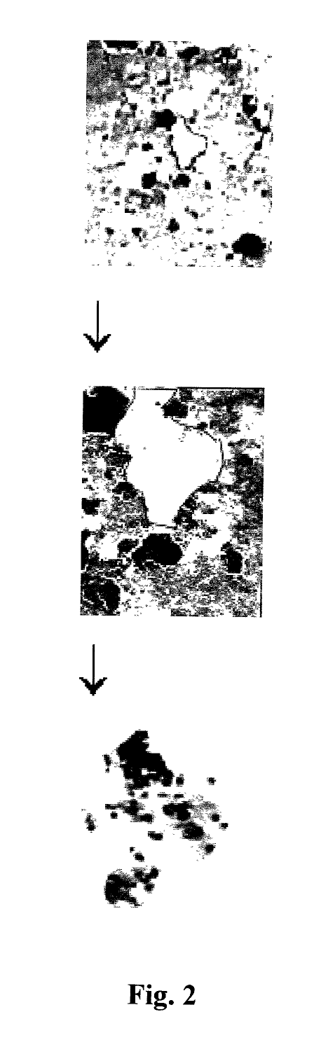

Breast ductal fluid specimens obtained by nipple aspirate or ductoscopy, as described above, were rinsed into containers holding 3% polyethylene glycol in denatured alcohol (Shandon Lipshaw, Pittsburgh, Pa.). The fixed specimens were then cytocentrifuged onto glass slides. Thereafter, the cytospin specimens were laser dissected following the standard protocol of Emmert-Buck, M. R. et al (Science, 274:998-1001, 1996), modified as necessary, with either a PixCell I or II LCM system (Arcturus Engineering, Mountain View, Calif.) for collection for a pure population of BECs, thereby eliminating heterogeneity concerns. (FIG. 2).

RNA Extraction

The total RNA from each population of laser captured cells was independently extracted by means of a modification of the RNA microisolation protocol as described by Emmert-Buck, M. R. et al (Science, 274:998-1001, 1996). Briefly, the transfer film and adherent cells were incubated with guanidinium isothiocyanate buffer at room temperature, extracted with phenol/chloroform/isoamyl alcohol, and precipitated with sodium acetate and glycogen carrier (10 μg/pI) in isopropanol. After initial recovery and resuspension of the RNA pellet, a DNase step was performed for 2 hr at 37° C. using 10 units of DNase (GenHunter, Nashville, Tenn.) in the presence of 10 units of RNase inhibitor (Life Technologies, Inc., Gaithersburg, Md.), followed by reextraction and precipitation. The pellet was then resuspended in 27 μl of RNase-free H₂O.

aRNA Amplification and Labeling

In specimens having insufficient cellularity to directly perform microarray analysis, aRNA amplification was performed. The aRNA amplification method employed was a linear rather than an exponential amplification (in contrast to RT-PCR, for example), and as a result better retained the original mRNA abundance information through the amplification process. A single round of aRNA amplification is unlikely to significantly bias the relative mRNA species abundance in a given sample, and is a convenient means to permit microarray analyses of samples of approximately 1-50 ng starting RNA. One round of amplification is thought to amplify the RNA approximately 2000-fold; a second round, one million fold. Where necessary, aRNA amplification of the RNA was performed using the protocol previously described by Brooks-Kayal, A. R. et al (Nat. Med.1998 October;4(10):1166-72).

Briefly, first-strand cDNA synthesis was done by incubating a solution containg deoxynucleotide triphosphates (dNTPs), avian myeloblastosis virus (AMV) reverse transcriptase, and T7-oligo-d(T) with total RNA at 42° C. for 60-90 minutes. After phenol-chloroform extraction and ethanol precipitation with E. coli tRNA as carrier, double-stranded DNA was made by incubation at 14° C. for 14-18 hr with dNTPs, T4 DNA polymerase and the Klenow fragment of DNA Polymerase I. The single-stranded hairpin loop was removed with S1 nuclease, the ends of the double-stranded template were blunted with T4 DNA polymerase and the Klenow fragment of DNA Polymerase I at 37° C. for 2 hr, then cDNA was drop-dialyzed against RNAse-free water to remove unincorporated dNTPs. The cDNA recovered from the filter was used for synthesis of amplified RNA (aRNA) in a solution containing Tris (pH 7.4), NaCl, MgCl2, and dithiothreitol, with addition of dNTPs, α-[³²P]-CTP, RNAasin, and T7 RNA polymerase at 37° C. for 4 hr. aRNA was then phenol-chloroform extracted, ethanol precipitated, resuspended in DEPC H₂O, and stored at −80° C. If necessary aRNA was then synthesized again into a single-stranded cDNA template for a second round of amplification.

Array Preparation

Microarrays are printed using a Hamilton 2000 with a 4 pin (or similar) printer, narrow gauge print head and home-written C++ macros for 16 well slide or 96 well plate printing: 150 μM, 300 μM on center. Duplicate copies of cDNA probes along with a series of housekeeping genes, including β-actin, GAPDH and alignment markers are printed onto exposysilane surfaces. The microarrays are stable at 4° C. until their use.

Hybridization

The labeled RNA is denatured and hybridized to the cDNA microarray as follows: the arrays are pre-hybridized at 42° C. in a roller oven (Hybaid, Midwest Scientific, St. Louis, Mo.) with 1.0 μg/ml poly-dA (Research Genetics) and 1.0 μg/ml Cot1 DNA (BRL/Life Technology) in 5 ml in Microhyb solution (Research Genetics) for at least 2 hours. After overnight hybridization with the radiolabeled targets, the arrays are washed twice at 50° C. in 2×SSC, 1% SDS for 20 minutes, and once at room temperature in 0.5×SSC, 1% SDS for 15 minutes. The arrays are then exposed overnight to a Packard screen and scanned at 50-micron resolution in a Phosphorimager instrument (Cyclone Instrument from Packard, Meriden, Conn.). Each hybridization is performed in duplicate. After each hybridization, the arrays are stripped by boiling in 0.5% SDS solution and scanned for residual leftover hybridization.

Image Analysis

The tiff images resulting from the phosphorimager are directly imported into the image analysis software Pathways (Research Genetics, Inc.). The software uses control spots present throughout the filter to align the images and performs autocentering, which aligns and centers well-shaped spots and deforms the calculated grid around spots that have a high confidence factor. When comparing two images, the software normalizes the two different hybridizations on the basis of the average total intensity on each filter. The software locates, calculates, and stores each cDNA spot intensity from each tiff file and simultaneously compares two different normalized tiff images. The differential expression ratios represent the average of two independent experiments.

Protein Microarrays for Detection and Quantitation of Specific Proteins in Breast Ductal Fluid

Preparation of Protein Solutions

Breast ductal fluid, obtained by nipple aspirate or ductoscopy, were used for preparing target proteins. Specimens were collected in 50 μl capillary tubes (generally 1-5 μl per tube). For extraction, the portion of the capillary containing the sample were introduced into a 1.7 ml eppendorf tube and 100 μl of a 0.1 M pH 8.0 sodium carbonate buffer was added. The cappilary was then crushed by using a glass rod and the mixture was vortexed to disperse the sample. The mixture was then centrifuged at 14,000 g for 5 min and the supernatant used in protein analysis without further dilution.

NHS-ester activated Cy3 and Cy5 solutions (Amersham PA23001 and PA25001) were prepared in a 0.1 M pH 8.0 sodium carbonate buffer. The protein and dye solutions were mixed together so that the final protein concentration was 0.2-2 mg/ml and the final dye concentration was 100-300 M. Normally approximately 15 μg protein was labeled per array. The reactions were allowed to sit in the dark for 45 min and then quenched by the addition of a tenth volume 1 M pH 8 Tris base (a 500-fold molar excess of quencher). The reaction solutions were brought to 0.5 ml with PBS and then loaded into microconcentrator spin columns (Amicon Microcon 10) with a 10,000 Da molecular weight cutoff. After centrifugation to reduce the volume to approximately 10 μl (approximately 20 min), a 3% non-fat milk blocking solution was added to each Cy5-labeled solution such that 25 μl milk was added for each array to be generated from the mix. (The milk was first spun down as above.) The volume was again brought to 0.5 ml with PBS and the sample again centrifuged to ˜10 μl. The Cy3-labeled reference mix was divided equally among the Cy5-labeled mixes, and PBS was added to each to achieve 25 μl for each array. Finally, the mixes were filtered with a 0.45 m spin filter (Millipore) by centrifugation at 10,000×g for 2 min.

Preparation of Arrays

Antibodies provided in glycerol solutions are transferred to a glycerol-free, phosphate-buffered saline (PBS) solution (137 mM NaCl, 2.7 mM KCl, 4.3 mM Na₂HPO₄, 1.4 mM KH₂PO₄, pH 7.4) using a BioRad Biospin P6 column. Antibody solutions are prepared at 0.1-0.3 mg/ml in 384-well plates, using approximately 4 μl per well. A robotic arrayer spots the protein solutions in an ordered array onto poly-L-lysine coated microscope slides at a 375 μm spacing using 16 steel tips. The coated slides are prepared as described in Eisen M B et al., Methods Enzymol, 303:179-205, 1999 or purchased from CEL Associates (Houston, Tex.). Briefly, glass microscope slides are cleaned in 2.5 M NaOH for 2 hr, rinsed thoroughly in ultra-pure H₂O, soaked for 1 hour in a 3% poly-L-lysine solution in PBS, rinsed in ultra-pure H₂O, spun dry, and further dried for 1 hr at 80° C. in a vacuum oven. The resulting microarrays are sealed in a slide box and stored at 4° C. The location of the array of spots is delineated on the back of the arrays with a diamond scribe (the spots disappear after washing). The arrays are rinsed briefly in a 3% non-fat milk/PBS/0.1% Tween-20 solution to remove unbound protein. They then are transferred immediately to a 3% non-fat milk/PBS/0.02% sodium azide blocking solution and allowed to sit overnight at 4° C. The milk solution is first spun for 10 min at 10,000×g to remove particulate matter. Excess milk is removed in three room temperature PBS washes of 1 min each, with the arrays remaining in the final wash until application of the probe solution.

Detection

Each microarray is removed individually from the PBS wash, and excess liquid is shaken off. Without allowing the array to dry, 25 μl dye-labeled protein solution is applied to the surface within the marked boundaries. A 24×30 mm cover slip is placed over the solution. The arrays are sealed in a chamber with an under-layer of PBS to provide humidification, after which they sit at 4° C. for 2 hr. Then the arrays are dunked briefly in PBS to remove the protein solution and the cover slip and allowed to rock gently in PBS/0.1% Tween-20 solution for 20 min. The arrays are subsequently washed twice in PBS for 5-10 min each and twice in H₂O for 5-10 min each. All washes are at room temperature. After spinning to dryness in a clinical centrifuge equipped with plate carriers (Beckman), the arrays are scanned in an Axon Laboratories (Palo Alto, Calif.) scanner using 532 nm and 635 nm lasers.

Analysis

The relative concentration of each protein in two separate dye-labeled pools is determined by comparing the fluorescence intensities in the Cy3- and Cy5-specific channels at each spot. The location of each analyte spot on the array is outlined using the gridding software GenePix (Axon Laboratories, Palo Alto, Calif.) and ScanAlyze. The background, calculated as the median of pixel intensities from the local area around each spot, is subtracted from the average pixel intensity within each spot. The background-subtracted values in the red channel are multiplied by a normalization factor to correct for detection differences in the two channels.

The normalization factor is found by comparing the red/green ratios of three to four well-behaved antibodies or antigens, which served as internal standards, to the ratio of the known concentrations. A factor is calculated which, when multiplied with the signal in the red channel, minimized the difference between the ideal and observed red/green ratios. A separate normalization factor is calculated for each array. To normalize the ratios for the antigens that are used in calculating the factor, a separate factor is used in which that particular antibody is dropped from the calculation (that is, a spot is never used to normalize itself). Finally, the ratios of the background-subtracted, normalized signal intensities are calculated to estimate the relative concentrations between proteins in the separately labeled pools.

Proteomic Analysis of Nipple Aspirate Fluid to Detect Proteins Associated with Breast Cancer

Material and Methods

NAF was collected using a modified breast pump (Sauter et al, Cancer Epidemiol Biomarkers Prev 1996, 5:967-970) and coded so that all analyses were blinded. Specimens were collected from women 35-81 years old scheduled to undergo surgery for a suspected malignancy in the aspirated breast, as well as from women without evidence of disease (Table 3). NAF was diluted with Tris buffer to a concentration of 3.6 mg total protein ml⁻¹ Tris buffer. Proteome analysis with a SELDI PBSII system (Ciphergen Biosystems, Fremont, Calif., USA) was carried out on three chips—normal phase (NP), hydrophobic (H4) and anion exchange (SAX)—using 1 ml of NAF. The chromatographic surfaces of these ProteinChips allowed the capture of generic proteins (NP), proteins having exposed hydrophobic surfaces (H4), and proteins binding by anion exchange (SAX). NP and SAX chips were washed first with binding buffer containing detergent, then with detergent-free buffer and water to remove nonspecific proteins. H4 chips were loaded with 10% acetonitrile, then washed with this solvent and water.

Results

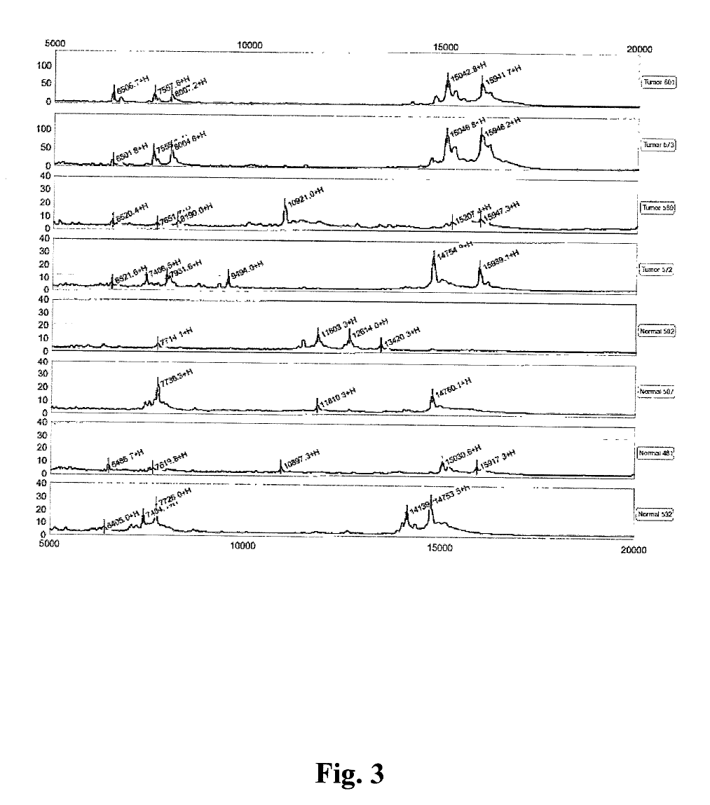

A representative ProteinChip SELDI MS analysis of NAF demonstrates discrete peaks (FIG. 3) at 6,500 and 8,000 Da from three of four tumor bearing women, which peaks are not present or are present at a reduced level in NAF from women with normal breast tissue. A protein is considered expressed if the value assigned by SELDI software had a signal-to-noise ratio ≧3:1. Protein peaks from different samples were judged similar if the values in Da were within 0.05% of each other. FIG. 3 also demonstrates a distinct peak at 15,940 Da in four out of four NAF samples from women with breast cancer which peak is not observed in NAF from normal subjects. Two larger proteins (a broad-based peak centered at 28,100 Da and a peak at 31,770) also were identified in a high percentage of women with breast cancer but were absent or present at a reduced level in NAF from normal women.

Percentages of positively expressed proteins in NAF from women with and without cancer and odds ratios (ORs) with 95% confidence intervals (C is) are listed in Table 4. Because of the small sample size, all estimates and P-values were computed using exact methods (LogXact 4.0, Cytel Software Corporation, Cambridge, Mass., USA). Protein peaks at 15,940 Da in all chips, at 8,000 Da in the H4 and NP chips, at 6,500 Da in the H4 and SAX chips, and at 28,100 Da in the NP chip were expressed significantly more often in NAF samples from subjects with breast cancer than from normal subjects (Table 4). For each chip, maximum sensitivity and specificity were achieved with the single protein peak: 15,940 Da in the H4 and NP chips, and 6,500 Da in the SAX chip (Table 4, bolded rows). These proteins were separately evaluated in multivariable logistic regression models controlling for age, race, past pregnancy, and past use of oral contraceptives. The adjusted odds ratios were: 29.2 (95% Cl:4.44 to ∞, P<0.001) for the 15,940 Da protein using the H4 chip, 27.3 (95% Cl:3.93 to ∞, P<0.001) for the 6,500 Da using the SAX chip, and 18.7 (95% Cl:2.52 to 6.36, P=0.002) for the 15,940 Da using the NP chip.

Discussion

Of the differentially expressed proteins identified using SELDI MS, the 6,500 and 15,940 Da proteins are particularly striking since they were detected in a high percentage of subjects with breast cancer but in no one (6,500 using the SAX chip and the 15,940 using the NP chip) or very few women without breast cancer. The identity of the 6,500 Da protein is not known but may represent epithelin, while the 8,000 Da peak may be mammaglobin. Mammaglobin has been reported to be a breast cancer marker. The identity of the 15,940 protein has not been determined. The 31,770 peak is most likely a dimer of the 15,940 protein, while the identity of the 28,100 Da peak may represent one or more members of the kallikrein family.

In conclusion, proteomic analysis of NAF from subjects with and without breast cancer identified five differentially expressed proteins. The most sensitive and specific proteins were 6,500 and 15,940 Da, found in 75-84% of samples from women with cancer but in only 0-9% of samples from normal women. While current tools to detect breast cancer are widely available and clinically effective, they have limited sensitivity and specificity. Analysis of NAF proteins secreted from the breast epithelium increases the ability to detect breast cancer at its earliest stages.

TABLE 3
Patient Demographics

| Menopausal status | Cancer | No cancer |
|---|---|---|
| Pre | 9 | 10 |
| Post- | 11 | 3 |
| Race | | |
| While | 17 | 9 |
| Nonwhite | 3 | 4 |
| Age | | |
| Median | 52 | 44 |
| Range | 33-81 | 29-55 |
| Ever pregnant | | |
| Yes | 17 | 8 |
| No | 3 | 5 |
| Median age (years) at 1st pregnancy | 27 | 21 |
| Ever used birth control pills | | |
| Yes | 11 | 8 |
| No | 9 | 5 |
| Ever used hormone replacement medications | | |
| Yes | 6 | 1 |
| No | 14 | 12 |

TABLE 4
Protein expression profile in NAF (by size) obtained from normal
women and from women with breast cancer, using three different
SELDI ProteinChips (NP, SAX, and H4)

| | Positive | | Positive | | | |
|---|---|---|---|---|---|---|
| Protein | in tumor | | in normal | | | |
| Chip peak | (sensitivity) | | (I-specificity) | | OR (95% CI) | P-value |
| NP | | | | | | |
| 6,500 Da | 12/19 | 63% | 3/11 | 27% | 4.33 (0.72, ∞) | 0.128 |
| 8,000 Da | 15/19 | 79% | 1/11 | 9% | 31.73 (3.11, 28 ) | <0.001 |
| 15,940 Da | 16/19 | 84% | 1/11 | 9% | 43.10 (4.01, ∞) | <0.001 |
| 28,100 Da | 8/19 | 42% | 0/11 | 0% | 9.61 (1.47, ∞) | 0.026 |
| 31,770 Da | 6/19 | 32% | 0/11 | 0% | 6.26 (0.77, ∞) | 0.091 |
| SAX | | | | | | |
| 6,500 Da | 15/20 | 75% | 0/12 | 0% | 54.67 (7.26, ∞) | <0.001 |
| 8,000 Da | 4/20 | 20% | 0/13 | 0% | 2.91 (0.24,160) | 0.661 |
| 15,940 Da | 11/20 | 55% | 0/13 | 0% | 19.04 (2.61, ∞) | 0.002 |
| 28,100 Da | 8/20 | 40% | 2/13 | 15% | 3.53 (0.53, 41.3) | 0.264 |
| 31,770 Da | 0/20 | 0% | 0/13 | 0% | n/e | |
| H4 | | | | | | |
| 6,500 Da | 13/20 | 65% | 2/13 | 15% | 9.44 (1.46,111) | 0.013 |
| 8,000 Da | 12/20 | 60% | 2/13 | 15% | 7.71 (1.20, 90.0) | 0.027 |
| 15,940 Da | 16/20 | 80% | 0/13 | 0% | 72.02 (9.32, 00) | <0.001 |
| 28,100 Da | 5/19 | 26% | 0/12 | 0% | 5.63 (0.69,∞) | 0.116 |
| 31,770 Da | 1/19 | 5% | 0/12 | 0% | 0.680.02, ∞) | 1 |

Prostate-Specific Antigen and Insulin-Like Growth Factor Binding Protein-3 in Nipple Aspirate Fluid are Associated with Breast Cancer

PSA is made by both normal and malignant human breast tissue. It has previously been reported that PSA is detectable in NAF, and low levels have been associated with breast cancer (Sauter ER et al., Cancer Epidemiol Biomarkers Prev 1996;5(12):967-70). It was not found, however, that PSA was useful in predicting the presence of residual disease in women after breast biopsy demonstrated cancer with positive or indeterminate margins (Sauter E R et al., Br J Cancer 1999;81(7):1222-7). PSA is a chymotrypsin-like protease that is thought to cleave IGFBP-3 at a specific tyrosine residue (Tyr-159), leading to fragmented IGFBP-3, or BP3-FR. BP3-FR has been found in a variety of body fluids from normal subjects, including lymph and serum and seminal plasma. BP3-FR inhibits the mitogenic effects of IGF-1. Multiple studies have confirmed that fragmented IGFBP-3 is biologically active. Thus, the interactions of PSA, IGFBP-3 and BP3-FR appear to play an important role in IGF-1 regulation. The Inventor recently proposed a hypothetical model of IGF-1 regulation that may help to explain how these molecules interact:

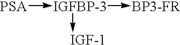

Materials and Methods

Subjects. NAF specimens from 175 subjects and serum specimens from 215 subjects aged 30-80 years were collected between January 1995 and July 1999. In some cases, more than one specimen was collected from an individual, with each specimen collected on a different day. If multiple results were available for an individual, only the median value was used for statistical analysis. Subjects included women with all stages of risk, from no risk factors for breast cancer (other than gender) to those with recently diagnosed carcinoma of the breast. Subjects were categorized as either (A) cancer: those with newly diagnosed, biopsy-proven ductal carcinoma in situ (DCIS) or invasive carcinoma (IC); or (B) non-cancer: no evidence of DCIS or IC. For subjects with cancer, NAF was collected from the breast with active disease.

Aspiration Technique. Nipple fluid was aspirated using a modified breast pump as described in Sauter ER et al, Cancer Epidemiol Biomarkers Prev 5:967-970, 1996. The breast nipple was cleansed with alcohol; the plunger of the aspiration device was withdrawn to the 7 ml level and held for 15 sec. Fluid in the form of droplets was collected in capillary tubes. The quantity of fluid varied from 1 to 200 μl.

PSA, IGFBP-3, BP3-FR. Specimen preparation (NAF): Every NAF sample collected was suitable for evaluation. In general, a sample was used to measure total protein and each one of the markers (PSA, IGFBP-3, or BP3-FR). Samples were collected in 50 μl capillary tubes (generally 1-5 μl per tube). For extraction, the portion of the capillary containing the sample was introduced into a 1.7 ml eppendorf tube and 100 μl of a 0.1 mol/l solution of sodium bicarbonate (pH 7.8) was added. The capillary was then crushed by using a glass rod and the mixture was vortexed to disperse the sample. The mixture was centrifuged at 14,000 g for 5 min and the supernatant was used without further dilution.

Specimen preparation (serum): A volume of 8 ml of blood was collected and the serum separated from the cellular fraction.

Specimen analysis (NAF and serum): The NAF samples varied both in their total protein concentration and in the volume in the capillary used for marker analysis. For this reason, the total protein concentration and the degree to which the NAF was diluted prior to analysis was controlled. Total protein was measured using the bicinchoninic acid method (Pierce Chemical Co., Rockford, Ill.). PSA was analyzed using a highly sensitive technique as described in Sauter E R et al., Cancer Epidemiol Biomarkers Prev 1996;5(12):967-70. Briefly, this procedure combines a time-resolved immunofluorometric assay (TRIFA) with two monoclonal antibodies and has a detection limit of 1 ng/l. IGFBP-3 was measured according to the manufacturer's instructions with a commercially available enzyme-linked immunosorbent assay (ELISA) from Diagnostic Systems Laboratories, Webster, Tex. This assay is based on a two-site immunoenzymatic principle with a polyclonal antibody used for capture and an enzyme-labeled polyclonal antibody used for detection. BP3-FR was measured with an immunoenzymatic assay based on a monoclonal capture antibody and another monoclonal detection antibody labeled with horseradish peroxidase. This assay has been previously described in Elmlinger M W et al., Pediatr Res 1999;46(1):76-81. The assay was calibrated with intact recombinant IGFBP-3.

Serum IGF-1. Specimen collection: A volume of 8 ml of blood was collected and the serum separated from the cellular fraction.

Specimen analysis: Serum IGF-1 concentration was measured with an immunoenzymatic assay commercially available from Diagnostic Systems Laboratories. The assay is based on a monoclonal capture antibody and a monoclonal detection antibody labeled with horseradish peroxidase. A non-extractive protocol was used, following the manufacturer's recommendations. The same method was used to measure IGF-1 in NAF but the concentration of the protein in the NAF samples was too low to detect.

Statistical Analysis. Women were included in the cancer group if they had known DCIS or IC, and in the non-cancer group if they did not have either of these diagnoses. Because menopausal status has been shown to influence the expression of a variety of breast cancer markers, results were analyzed overall and by menopausal status (pre/peri-versus postmenopausal). The Wilcoxon two-sample test was performed to compare the levels of NAF and serum markers in the cancer and non-cancer groups. The exact version of the Wilcoxon test was used to verify all significant P-values. All tests of statistical significance were two sided.

Multivariate logistic regression analyses were then performed controlling for covariates potentially associated with breast cancer. The following covariates were considered: race, age, menopausal status, age at menarche, age when the first child was born, birth control usage and hormone replacement therapy. Age, menopausal status, and age at menarche were associated with the odds of cancer in some of the models, and generally improved the fit of all the models. The logistic regression models included these three factors, even if they were not statistically significant, and one or more of the markers therefore were analyzed. The Wald test was used to assess the significance of variables in the model, while the profile likelihood method was used to compute the confidence intervals for the odds ratios (McCullagh P et al., Generalized Linear Models, New York: Chapman & Hall, 1991).

Although logistic regression models routinely estimate the increase in odds per one unit of increase in the covariate, because this increase was not biologically noteworthy for some of the covariates, the odds ratios corresponding to 5 and 10 U increase in age and NAF IGFBP-3 were computed as respectively the 5th and 10th power of the odds ratio per unit increase. NAF PSA was analyzed on the log scale, that is, the percent of increase in NAF PSA corresponded to the absolute increase in In(NAF PSA). The odds ratios corresponding to the 50 or 100% increase in NAF PSA were computed as the increase in odds corresponding to respectively In(1.5) or In(2) increase in the natural log transformed NAF PSA.

Logistic models combining NAF and serum markers were not feasible since only 21 women had both types of markers measured. Empirical logits were used to verify that the relationship between the log odds and each original continuous marker was approximately linear and to select the appropriate transformations for the marker variables, when necessary. Based on this analysis in the logistic models log transformed NAF and serum PSA (adding 0.5 to the original value in order to accommodate multiple zero values) and squared serum IGFBP-3 and BP3-FR and the original values of NAF IGFBP-3 and BP3-FR were used. All markers were included as continuous variables in the logistic regression models. The following outlier data were excluded from the logistic analyses: serum PSA=7,335 and 219 ng/l, NAF PSA=25,016 and 17,191 ng/g, and NAF IGFBP-3=583 μg/mg. In order to define potential marker cut points associated with breast cancer, classification and regression trees (CART) analysis was performed on the data including significant covariates and markers from the corresponding logistic regression models. The data were analyzed using SAS 8.0 (SAS Institute Inc., Cary, N.C.) and classification and regression trees (CART, San Diego, Calif.: Salford Systems, 1997).

Results

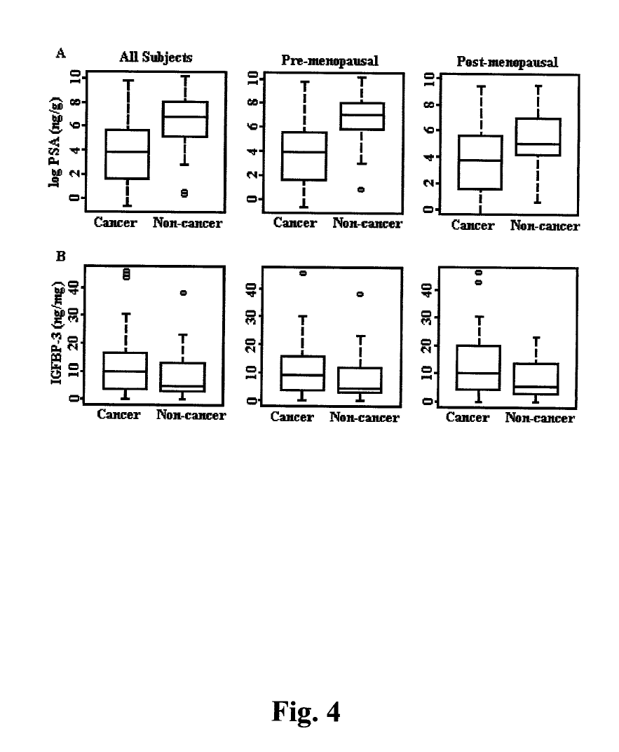

NAF from 98% of enrolled subjects was obtained. Table 5 shows the results of the Wilcoxon two-sample tests that were performed to compare the levels of the markers in the cancer and non-cancer groups. Considering all subjects, median NAF PSA was significantly (P<0.001) higher in non-cancer subjects (852 ng/g) than in subjects with cancer (46 ng/g), while median NAF IGFBP-3 was significantly lower (P=0.023) in non-cancer subjects (4.7 ng/mg) than in subjects with cancer (9.8 ng/mg). NAF PSA and IGFBP-3 results were then analyzed by menopausal status. In pre-menopausal women, median NAF PSA was significantly higher (P<0.001) in non-cancer subjects (1112 ng/g) than in subjects with cancer (49 ng/g) (Table 5, FIG. 4A). In postmenopausal women, median NAF PSA was significantly higher (P=0.010) in non-cancer subjects (154 ng/g) than in cancer subjects (41 ng/g). Though both pre- and post-menopausal non-cancer subjects had lower median levels of IGFBP-3 than subjects in the cancer group (pre-menopausal: 4.5 versus 8.9 ng/mg; postmenopausal: 5.7 versus 10 ng/mg), these differences did not reach statistical significance (Table 5, FIG. 4B). NAF levels of BP3-FR were not different between the groups, whether considering all subjects or subjects divided by menopausal status.

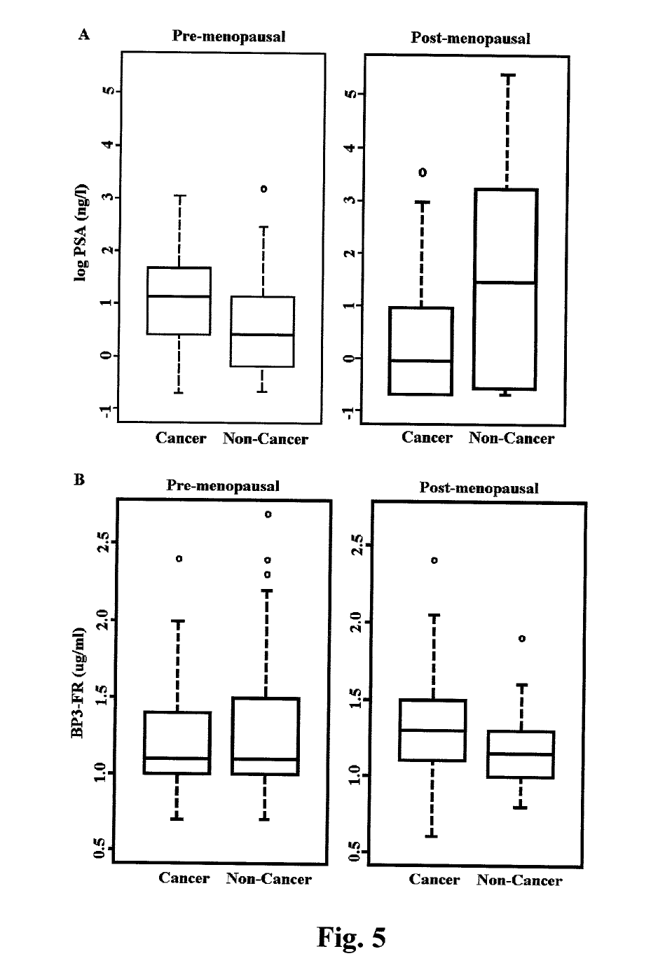

Whether considering all subjects or pre-menopausal subjects only, no serum marker was significantly different in the cancer and non-cancer groups. On the other hand, in postmenopausal women median serum PSA was significantly higher (P=0.034) in non-cancer subjects (3.8 ng/l) than in subjects with cancer (0.5 ng/l) (Table 5, FIG. 5A), and median serum BP3-FR was significantly lower (P=0.026) in non-cancer subjects (1.2 μg/ml) than in subjects with cancer (1.3 μg/ml) (Table 5, FIG. 5B).

TABLE 5
Medians of PSA, IGF-1, IGFBP-3, BP3-FR levels in women with and
without breast cancer^{a}

| | | | Non-caner | | Cancer | | |
|---|---|---|---|---|---|---|---|
| | All subjects | Overall median | Subjects | Median | Subjects | Median | P-value |
| Both pre-and postmenopausal women^{b} | | | | | | | |
| NAF | | | | | | | |
| PSA (ng/g) | 171 | 133 | 58 | 852 | 113 | 46 | <0.001 |
| IGFBP-3 (ng/mg) | 126 | 7.4 | 42 | 4.7 | 84 | 9.8 | 0.023 |
| BP3-FR (ng/mg) | 111 | 29 | 34 | 30 | 77 | 28 | 0.975 |
| Serum | | | | | | | |
| PSA (ng/l) | 89 | 1.1 | 40 | 1.5 | 49 | 0.9 | 0.071 |
| IGF-1 (ng/ml) | 151 | 166 | 89 | 173 | 62 | 157 | 0.125 |
| IGFBP-3 (μg/ml) | 198 | 3.2 | 82 | 3.2 | 116 | 3.2 | 0.838 |
| BP3-FR(pg/ml) | 198 | 1.2 | 82 | 1.1 | 116 | 1.2 | 0.071 |
| Pre-menopausal women only^{b} | | | | | | | |
| NAF | | | | | | | |
| PSA(ng/g) | 86 | 319 | 41 | 1112 | 45 | 49 | <0.001 |
| IGFBP-3 (ng/mg) | 61 | 5.9 | 2 | 4.5 | 32 | 8.9 | 0.102 |
| BP3-FR (ng/mg) | 50 | 32 | 22 | 31 | 28 | 37.5 | 0.914 |
| Serum | | | | | | | |
| PSA(ng/l) | 35 | 1.4 | 21 | 1 | 14 | 2.7 | 0.172 |
| IGF-1 (ng/ml) | 73 | 182 | 51 | 187 | 22 | 173 | 0.564 |
| IGFBP-3 (μg/ml) | 92 | 3.2 | 49 | 3.2 | 43 | 2.9 | 0.214 |
| BP3-FR (μg/ml) | 92 | 1.1 | 49 | 1.1 | 43 | 1.1 | 0.837 |
| Postmenopausal women only^{b} | | | | | | | |
| NAF | | | | | | | |
| PSA (ng/g) | 85 | 55 | 17 | 154 | 68 | 41 | 0.010 |
| IGFBP-3 (ng/mg) | 65 | 9.6 | 13 | 5.7 | 52 | 10 | 0.251 |
| BP3-FR (ng/mg) | 61 | 28 | 12 | 29 | 49 | 28 | 0.986 |
| Serum | | | | | | | |
| PSA (ng/l) | 46 | 0.6 | 11 | 3.8 | 35 | 0.5 | 0.034 |
| IGF-1 (ng/ml) | 68 | 152 | 28 | 152 | 40 | 152 | 0.356 |
| IGFBP-3 (μg/ml) | 99 | 3.2 | 26 | 3.2 | 73 | 3.4 | 0.667 |
| BP3-FR (μg/ml) | 99 | 1.3 | 26 | 1.2 | 73 | 1.3 | 0.026 |

^{a}IGF-1 results are only available from serum. Some subjects provided only NAF or serum, but not both.
^{b}The number of pre- and postmenopausal women might not add up to the total number because of occasionally missing data on menopausal status.

Logistic Regression Analyses of NAF and Serum Markers. Table 6 summarizes the results of the logistic regression models for NAF PSA (164 subjects) and NAF IGFBP-3 (119 subjects). NAF PSA was highly significant in predicting cancer in pre-(P<0.001) but not in post-menopausal women (P=0.089).

In pre-menopausal women for NAF PSA, the model implies that the odds of having cancer decrease 22.5% (95% Cl: 14.3 and 31.7%) with each 50% rise in PSA. If NAF PSA is doubled (an increase of 100%), the odds of having cancer decrease 35.3% (95% Cl: 23.2 and 47.8%). In post-menopausal women, the odds of having cancer decrease 8.2% (95% Cl:−0.9 and 17.4%) with a 50% rise in NAF PSA and 13.7% for each 100% increase in PSA (Cl:−1.6 and 27.9%).

NAF IGFBP-3 was a significant predictor of cancer in all women (P=0.031), and results did not depend on menopausal status. The model for NAF IGFBP-3 implies that the odds of having cancer increase 32.3% (95% Cl:6.5 and 76.7%) for every 5 ng/mg increase and 75% (95% Cl:13.4 and 212.2%) for every 10 ng/mg increase in this marker for women of all ages.

The model including both NAF PSA and NAF IGFBP-3 yielded a significant effect of NAF PSA, but a small and statistically insignificant effect of NAF IGFBP-3. Thus, if age, menopausal status, age at menarche, and NAF PSA are known, NAF IGFBP-3 contributed little additional information about the chances that a subject had breast cancer.

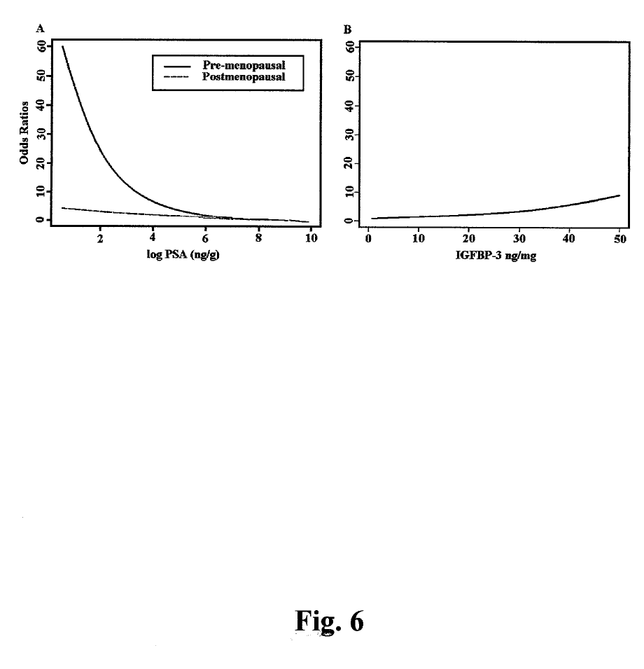

Models for the serum markers indicated that after controlling for age, menopausal status and age at menarche, no serum marker was significantly associated with the odds of a subject having cancer. The inverse association of NAF PSA with the odds of having breast cancer in pre- and post-menopausal women is illustrated in FIG. 6A. The odds are shown relative to a woman with a median NAF PSA value from the non-cancer pre-menopausal group (1112 ng/g). The direct association of NAF IGFBP-3 with the odds of breast cancer for both pre- and postmenopausal women is illustrated in FIG. 6B. Odds are shown relative to a woman with a median NAF IGFBP-3 value from the non-cancer group (4.72 ng/mg).

The ratios in NAF of IGFBP-3 to PSA and IGFBP-FR to PSA were considered, as well as IGF-1 to IGFBP-3, IGF-1 to PSA, IGFBP-3 to PSA, and IGFBP-FR to PSA in serum. In separate logistic regression models, controlling for age, menopausal status, and age at menarche, only the ratio of the IGFBP-FR to PSA in NAF from pre-menopausal women was significantly associated with breast cancer (P=0.004), although the ratio of the IGFBP-3 to PSA in NAF was of marginal statistical significance (P=0.083). There were 92 observations available for these models. None of the ratios in the serum was significantly associated with breast cancer, although due to missing data there were only 56 observations available for these models.

TABLE 6
Odds ratios and confidence intervals from the
logistic regression models

| Variable | Unit of increase | Odds ratio | Lower 95% Cl | Upper 95% Cl | P-value |
|---|---|---|---|---|---|
| Model with NAF PSA | | | | | |
| Age | 5 years | 1.564 | 1.164 | 2.160 | 0.004 |
| | 10 years | 2.445 | 1.335 | 4.665 | |
| Menopausal status | Post vs. pre | 0.099 | 0.008 | 1.093 | 0.064 |
| Age at menarche | 1 year | 1.050 | 0.966 | 1.129 | 0.245 |
| NAF PSA in pre- | | | | | |
| menopausal women | 50% | 0.775 | 0.683 | 0.857 | <0.001 |
| | 100% | 0.647 | 0.522 | 0.768 | |
| NAF PSA in | | | | | |
| postmenopausal women | | | | | |
| | 50% | 0.918 | 0.826 | 1.009 | 0.089 |
| | 100% | 0.863 | 0.721 | 1.016 | |
| Model with NAF IGFBP-3 | | | | | |
| Age | | | | | |
| | 5 years | 1.511 | 1.116 | 2.137 | 0.012 |
| | 10 years | 2.284 | 1.245 | 4.566 | |
| Menopausal status | Post vs. pre | 1.024 | 0.283 | 3.604 | 0.970 |
| Age at menarche | 1 year | 1.068 | 0.878 | 1.121 | 0.099 |
| NAF IGFBP-3 | 5 ng/mg | 1.323 | 1.065 | 1.767 | 0.031 |
| | 10 ng/mg | 1.750 | 1.134 | 3.122 | |

NAF IGFBP-3: the effect of NAF IGFBP-3 was not different by menopausal status.

Categorizing Risk Using Classification and Regression Trees (CART). Since only age, PSA, and the interaction between menopausal status and PSA were significant in the logistic regression model for NAF PSA, the cut points for PSA in the CART model included age and menopausal status. PSA was more discriminatory than age and menopausal status regarding risk. In the samples, 92% (65/71) of women with a PSA value (≦65 ng/g) had breast cancer, whereas analysis of women with PSA>65 was inconclusive (44/93, or 47% had cancer).

In the logistic regression model for NAF IGFBP-3, only age and IGFBP-3 were significant. The cut points for IGFBP-3 included only these variables in the CART model. In contrast to PSA, IGFBP-3 was less discriminatory than age regarding risk, and was not informative for women >60 years old. The 100% (8/8) of women younger than 60 with IGFBP-3>26 ng/mg had breast cancer. Analysis of women younger than 60 with IGFBP-3=26 was inconclusive (39/80 or 49% had cancer).

Discussion

No association between IGF-1 serum concentration and breast cancer was found, whether considering the entire group or divided by menopausal status.

A significant association between PSA levels in NAF and the odds of breast cancer was found, both for the group as a whole, and when divided by menopausal status. The odds of both pre- and postmenopausal women having breast cancer is low if their PSA is high. Moreover, it is noteworthy that the median PSA value was more than 22-fold greater in pre-menopausal women without breast cancer than in those with cancer but only 3.76-fold higher in postmenopausal women.

The odds of a woman having breast cancer changed most dramatically at the lowest PSA levels, especially for pre-menopausal women. For example, for a pre-menopausal woman with NAF PSA=200 ng/g, a 100 ng/g decrease to 100 ng/g increased her odds of cancer by 55%, a 150 ng/g decrease to 50 ng/g increased her odds of cancer by 139%, and a 195 ng/g decrease to 5 ng/g increased her odds of cancer by 916%. On the other hand, for a pre-menopausal woman with NAF PSA=1000 ng/g, a 100 ng/g decrease to 900 ng/g increased her odds of cancer only by 7% and a 200 ng/g decrease to 800 ng/g increased her odds of cancer only by 15%.

In an effort to better understand the association of members of the IGF-1 family with breast cancer, IGFBP-3 and BP3-FR also were analyzed. The median NAF level of IGFBP-3 was twice as high in women with breast cancer as in women without breast cancer (Table 5, FIG. 2B), a statistically significant difference. This degree of difference was similar in both pre- and post-menopausal women yet significance was lost when the data were separated, which may have been due to sample size. Consistently, in the logistic regression model, the effect of IGFBP-3 did not vary by the menopausal status.

Although serum PSA results were univariately associated with postmenopausal breast cancer, when age, menopausal status, and age at menarche were controlled, no serum marker was significantly associated with the odds of a subject having cancer. This implies that serum results are less reliable than NAF because they reflect the contribution of a variety of bodily organs.

In the logistic regression models, which considered a number of clinical covariates, higher levels of NAF PSA in pre-menopausal women improved the ability to predict whether or not they had breast cancer, whereas it did not in post-menopausal women. PSA was a powerful predictor in pre-menopausal women, more powerful than age, which has been shown to be highly associated with a woman's odds of having breast cancer (Scanlon E F et al., Breast Cancer, In: Holleb A I, Fink D J, Murphy G P, editors, Textbook of Clinical Oncology. Atlanta: The American Cancer Society, 1991. p. 177-93). The association of PSA with post-menopausal breast cancer was not significant in the logistic regression model. The sample size may have lacked sufficient power to detect a difference, as there were only nine postmenopausal subjects without breast cancer in the model. When no information is available on NAF PSA, IGFBP-3 also is helpful in predicting a woman's odds of having breast cancer using logistic regression. In the presence of PSA, IGFBP-3 is no longer significantly associated with a woman's odds of having breast cancer.

In secondary statistical analysis it was found that, controlling for age, menopausal status, and age at menarche, the ratio of BP3-FR to PSA in NAF from pre-menopausal women was significantly associated with breast cancer (P=0.004) and the ratio of IGFBP-3 to PSA in NAF was of marginal statistical significance (P=0.083). These results reflect the significant effect of PSA and do not seem to provide additional insight above and beyond the effect of PSA.

CART analysis was used to determine cut points below or above which one could identify women likely to have or be free of breast cancer. It was found that 92% of women in the sample with a PSA value (65 ng/g) had breast cancer, while 100% of women younger than 60 with IGFBP-3>26 ng/mg had breast cancer.

In summary, the data demonstrate that PSA levels in NAF are inversely associated with the presence of breast cancer, especially in pre-menopausal women. NAF IGFBP-3, an important binding protein of IGF-1, is significantly higher in women with breast cancer. Serum PSA and BP3-FR are associated with postmenopausal breast cancer. Using logistic regression it was determined that both NAF PSA and IGFBP-3 are helpful in identifying women with breast cancer, even controlling for clinical variables known to be associated with the disease.

The determination of cut points, which identified subjects at very high risk of having breast cancer, implies that NAF biomarkers can be evaluated and criteria established to identify women who have the disease.

What is claimed is:

1. A microarray comprising a plurality of oligonucleotide probes immobilized on a surface of a solid support, wherein
a) each different oligonucleotide probe is localized in a predetermined region of said surface; and
b) said each oligonucleotide probe selectively hybridizes to an mRNA or a nucleic acid derived therefrom, wherein said mRNA is a transcript of a gene, wherein changes in expression levels of said gene are associated with breast cancer.

2. The microarray of claim 1, wherein said gene or a gene product thereof is involved in apoptosis.

3. The microarray of claim 1, wherein said gene encodes an insulin-like growth factor.

4. A kit comprising the microarray of claim 1 and one or more reagents for use in the assay to be performed.

5. A method for detecting breast cancer in a patient comprising:
a) obtaining a nucleic acid sample from BECs of said patient, wherein said nucleic acid sample contains a population of mRNA or nucleic acids derived therefrom;
b) providing a microarray according to claim 1;
c) hybridizing said nucleic acid sample to said microarray to form hybrid duplexes between nucleic acids on said nucleic acid sample and said oligonucleotide probes on said microarray; and
d) determining differences in hybridization compared to that from a non-cancer subject wherein said differences in hybridization indicate breast cancer in said patient.

6. The method of claim 5, wherein said BECs are obtained from breast ductal fluid.

7. The method of claim 6, wherein said BECs are obtained from NAF.

8. The method of claim 6, wherein said BECs are obtained from mammary ductal irrigation.

9. A method for determining efficacy of a therapeutic in treating or preventing breast cancer comprising:
a) administering an appropriate amount of said therapeutic to a patient having breast cancer;
b) obtaining a nucleic acid sample from BECs of said patient, wherein said nucleic acid sample contains a population of mRNA or nucleic acids derived therefrom;
c) providing a microarray according to claim 1;
d) hybridizing said nucleic acid sample to said microarray to form hybrid duplexes between nucleic acids on said nucleic acid sample and said oligonucleotide probes on said microarray; and
e) determining differences in hybridization compared to that prior to the administration of said therapeutic wherein said differences in hybridization indicate the efficacy of said therapeutic.

10. The method of claim 9, wherein said BECs are obtained from breast ductal fluid.

11. The method of claim 10, wherein said BECs are obtained from NAF.

12. The method of claim 10, wherein said BECs are obtained from mammary ductal irrigation.

13. A microarray comprising a plurality of protein-capture agents immobilized on a surface of a solid support, wherein
a) each different protein-capture agent is localized in a predetermined region of said surface; and
b) said each protein-capture agent selectively hybridizes to a protein, wherein changes in quantities of said protein is associated with breast cancer.

14. The microarray of claim 13, wherein said protein-capture agents aptamers.

15. The microarray of claim 13, wherein said protein-capture agents are polypeptides.

16. The microarray of claim 13, wherein said protein is selected from erbB-1; HER-2/neu; hTERT; erbB-2; hst-1; KDR; int-2; cyclin D1, 2, 3; CEA; H-ras; K-ras; mammoglobin; PSA; IGFBP-1, -2, -4, -5, -6; hK2; IGFBP-3; or p53.

17. A kit comprising the microarray of claim 13 and one or more reagents for use in the assay to be performed.

18. A method for detecting breast cancer in a patient comprising:
a) obtaining a sample from breast ductal fluid of said patient, wherein said sample contains a population of proteins;
b) providing a microarray according to claim 13;
c) hybridizing said sample to said microarray to form hybrid duplexes between said proteins on said sample and said protein-capture agents on said microarray; and
d) determining differences in hybridization compared to that from a non-cancer subject, wherein said differences in hybridization indicate breast cancer in said patient

19. The method of claim 18, wherein said breast ductal fluid is NAF or mammary ductal irrigation.

20. A method for determining efficacy of a therapeutic in treating or preventing breast cancer comprising:
a) administering an appropriate amount of said therapeutic to a patient having breast cancer;
b) obtaining a sample from breast ductal fluid of said patient, wherein said sample contains a population of proteins;
c) providing a microarray according to claim 13;
d) hybridizing said sample to said microarray to form hybrid duplexes between said proteins on said sample and said protein-capture agents on said microarray; and
e) determining differences in hybridization compared to that prior to the administration of said therapeutic, wherein said differences in hybridization indicate the efficacy of said therapeutic.

21. The method of claim 20, wherein said breast ductal fluid is NAF or mammary ductal irrigation.

22. A method for identifying proteins associated with breast cancer comprising:
a) obtaining a sample from mammary ductal fluid of a patient having breast cancer;
b) providing a proteinchip array having a selectivity condition, wherein the selectivity condition is defined by an adsorbent and an eluant;
c) contacting said sample to said proteinchip array;
d) washing said proteinchip array with said eluant; and
e) detecting and quantifying proteins retained by said proteinchip array by a laser desorption/ionization time-of-flight mass spectrometer, wherein one or more said proteins differentially present on said proteinchip array compared to that from a non-cancer subject are identified as the proteins associated with breast cancer.

23. The method of claim 22, wherein said breast ductal fluid is NAF or mammary ductal irrigation.

24. A method for detecting breast cancer in a patient comprising:
a) obtaining a sample from mammary ductal fluid of said patient, wherein said sample comprises at least one breast cancer related protein;
b) providing a proteinchip array having a selectivity condition, wherein the selectivity condition is defined by an adsorbent and an eluant, wherein the selectivity condition allows said at least one breast cancer related protein be retained by said proteinchip array;
c) contacting said sample to said proteinchip array;
d) washing said proteinchip array with said eluant;
e) detecting and quantifying said at least one breast cancer related protein retained by said proteinchip array by a laser desorption/ionization time-of-flight mass spectrometer; and
f) determining differences in quantities of said at least one breast cancer related protein retained by said proteinchip array compared to that from a non-cancer patient, wherein any differences in quantities indicate breast cancer in said patient.

25. The method of claim 24, wherein said breast ductal fluid is NAF or mammary ductal irrigation.

26. A method for determining efficacy of a therapeutic in treating or preventing breast cancer comprising:
a) administering an appropriate amount of said therapeutic to a patient having breast cancer;
b) obtaining a sample from mammary ductal fluid of said patient, wherein said sample comprises at least one breast cancer related protein;
c) providing a proteinchip array having a selectivity condition, wherein the selectivity condition is defined by an adsorbent and an eluant, wherein the selectivity condition allows said at least one breast cancer related protein be retained by said proteinchip array;
d) contacting said sample to said proteinchip array;
e) washing said proteinchip array with said eluant;
f) detecting and quantifying said at least one breast cancer related protein retained by said proteinchip array by a laser desorption/ionization time-of-flight mass spectrometer; and
g) detecting differences in quantities of said at least one breast cancer related protein retained by said proteinchip array compared to that prior to the administration of said therapeutic, wherein any differences in quantities indicate the efficacy of said therapeutic.

27. The method of claim 26, wherein said breast ductal fluid is NAF or mammary ductal irrigation.
